(19) United States
(12) Patent Application Publication
(10) Pub. No.: US 2011/0126831 A1
(43) Pub. Date: Jun. 2, 2011

(54) PORTABLE ESSENCE VAPORIZER

(76) Inventor: Jorge Fernández Pernía, Burgos (ES)

(21) Appl. No.: 12/675,707

(22) PCT Filed: Aug. 14, 2008
(86) PCT No.: PCT/ES08/70167

§ 371 Date: Feb. 26, 2010

(30) Foreign Application Priority Data
Aug. 28, 2007 (ES) P 200702406

(51) Int. Cl. A61M 15/00; A61M 11/04

(52) U.S. Cl. 128/203.27

(57) ABSTRACT

The portable essence vaporizer according to the present invention comprises a heat source for heating up an air flow which is passed through a substance from which the essence is to be extracted and is supplied to the user, and it comprises a heat exchanger which provides heat generated by the heat source to the air flow, and a thermal insulation which insulates the exterior from the heat source and the exchanger, said insulation being preferably microporous and having monolithic structures of metal oxides, amorphous silica or glass filaments, and the heat exchanger comprising an open cell foam structure; the vaporizer provides a satisfactory extraction and at the same time is of reduced size and has reduced electrical consumption.

The present invention relates to a portable essence vaporizer that extracts an essence and supplies it to a user, comprising a heat source for heating an air flow which is passed through a substance from which the essence is to be extracted, and then is supplied to the user.

### BACKGROUND

In the state of the art and in the market, there are several known electrical vaporizing devices for substances such as tobacco, aromatic or medicinal plants, etc. These devices cause the selective evaporation of essences, flavors, aromas or other active ingredients contained in the previously mentioned substances. The vapor of the essences is extracted by heating the substance in its solid or liquid state to a temperature equal or higher than the evaporation point of the essence that is contained in said substance, causing it to change to a gaseous state and thereby turning into vaporized essence.

There are several methods used to apply heat to a substance in order to cause evaporation. The two main ones are: extraction with heating by conduction or extraction with heating by convection.

The method of heating with thermal conduction, such as the one used in the patent US 2003/0150451, involves applying heat to a surrounding surface that encircles the substance until the evaporation temperature of the essence is reached. One of its disadvantages is that the system has a big thermal inertia, so that when one wants to stop the evaporation, the surrounding surface has to be cooled down with its corresponding energy losses. Or alternatively, the substance has to be removed from the surrounding surface, making it burdensome for the user who has to do this operation repeatedly. Another inconvenience of this method is that the quantity of vapor extraction is independent of the flow inhaled by the user. This means that the user cannot control the amount of vapor extracted. The device evaporates the same quantity independently of whether there has been inhalation, not taking into account whether the user actually uses the vapor or not.

Vaporizers that use methods of extraction by thermal convection are based on the heating of air or other gas until a temperature equal or higher than the evaporation point of the essence is reached. The method of extraction by convection has been proven to give better results time wise than the methods of extraction by conduction since the amount of obtained vapor is directly proportional to the warm air flow going through the substance. This way, if the warm air does not pass through the substance, there is no evaporation. The advantage is that the user has control over the quantity of extracted vapor since it is proportional to the volume of warm air that passes over the substance during the inhalation process.

The advantages shown in the method of extraction by air or gas convection compared to the heating method by conduction make its use more recommendable.

A vaporizer known in the prior art is described in the patent with publication number U.S. Pat. No. 4,141,369, relating to an essence extracting vaporizer by means of forced thermal convection (heating of air or other gas until a temperature equal or higher than the essence's point of evaporation is reached). Said vaporizer uses a heat source to increase the temperature of the air which, when passing through the substance, causes the evaporation of its essence. And, in case of a substance made of combustible material, prevents its combustion with this same method; and it furthermore includes an air chamber to isolate the heat source from the casing of the vaporizer.

However, the air inside this known vaporizer is unable to reach high temperatures, especially if it has to be adapted to a portable size. This is due to the limited amount of heat that can be transferred to air, and to the huge dimensions the air chamber should have to avoid an excessive exterior temperature.

For this reason, this known device has a low performance and a poor extraction efficiency.

The purpose of the current invention is to provide a compact portable essence vaporizer of small dimensions in which air can reach high temperatures for a satisfactory extraction as well as having a good energy performance, and as a result, a reduced electricity consumption.

From this point on, the term substance will be used when to refer to the chemical compounds, both in the form of plant or solid or liquid extract, from which one wants to obtain essence or essence vapor (which is the term that will be used hereinafter for the product obtained through the extraction process).

### BRIEF DESCRIPTION

The vaporizer of the current invention extracts the essence of a substance placed in the vaporizer, through a heating method using thermal convection of air or another gas. The vaporizer can be used as an aroma or taste extractor for spices in the cooking process; as an extractor of odor in aromatherapy treatments for psychosomatic conditions; for the extraction of active ingredients and their medical use; as a vaporizer and inhaler of nicotine for tobacco users, reducing the risks which come with the consumption through combustion.

The current invention relates to a portable essence vaporizer that extracts an essence and supplies it to a user, comprising a heat source for heating up an air flow that is passed through a substance, of which the essence is to be extracted, and is supplied to the user, wherein it comprises a heat exchanger which supplies heat generated by the heat source to the air flow, and a thermal insulation which insulates the heat source and the heat exchanger form the exterior.

Thanks to these features, the essence extraction can be performed at higher air temperatures than in other known devices, with a better energy performance and with reduced dimensions that make the device easy to handle, easy to transport, etc.

In some embodiments the insulation comprises a nanoporous material with a pore size smaller than 50 nm; preferably this nanoporous insulating material is microporous, with a pore size smaller than 2 nm.

The insulation can include monolithic structures of metal oxides, amorphous silica and glass filaments. In the context of the current invention, the term “glass filaments” refers to filaments, of any material, which are crystallized in an amorphous or vitreous manner.

The structure and components give the material a very low thermal conductivity, even at high temperatures, allowing for a reduction of the thickness of the insulation and consequently of the size of the vaporizer, as well as contributing to a very low contraction when cooling down. Additionally, they limit the external temperature of the vaporizer so that it can be handled without risk of burns or degradation of the outer casing. Furthermore, they reduce the energy losses so as to make the system perform with optimal electricity consumption.

In embodiments of the invention the heat exchanger consists of an open cell foam structure. Thanks to the high specific surface area of these materials, the exchanger conducts heat better and faster to the air surrounding it, improving the performance of the vaporizer. Consequently, the user can extract a high quantity of essence vapor in less time and with less inhaled volume.

In some embodiments, the open cell foam structure has a specific surface area of between 50 and 200 cm²/cm³.

The open cell foam structure can be metallic or ceramic. The ceramic foam structure provides a higher specific heat capacity than metal, whereas the metallic structure offers better thermal conductivity from the heat source to the heat exchanger.

Preferably the heat exchanger comprises a tubular shaped piece, the heat source being provided at least partly within the tubular shaped piece.

Also preferably, the thermal insulation includes a tubular shaped element that surrounds the heat exchanger at least partially, the whole being provided within the casing which will be handled by the user.

In embodiments of the invention, the vaporizer comprises a duct for air flow circulation, said duct including an extraction chamber arranged downstream from the heat source and used for the purpose of housing the substance.

In one embodiment, the vaporizer also comprises a humidifier placed in such a way as to allow the air flow to go through it.

During the essence extraction process, such humidifier causes the warm and dry air that circulates from the exchanger to the exit of the vaporizer to absorb enough humidity to make the inhalation process more pleasant for the user in the case of essence extraction through suction.

The humidifier can be placed downstream from the extraction chamber so that the air flow goes through it after the essence extraction. Alternatively, the humidifier can be place upward of the extraction chamber, so that the air flow goes through it before the essence extraction.

Preferably, the vaporizer includes an inhalation nozzle through which the user can inhale to cause the air flow going through the substance from which the essence is to be extracted and subsequently being inhaled by the user.

In some embodiments, the nozzle comprises a first branch through which the air flow that the user inhales circulates, and at least a second branch through which the air flow that the user exhales circulates.

According to an advantageous embodiment, the vaporizer furthermore comprises a vapor collector whose purpose it is to filter the exhaled vapor by the user after performing an essence extraction. The vapor collector can comprise activated carbon.

This way, the waste air exhaled by the user who has inhaled the essence is filtered through the vapor collector and, through the activated carbon, filtering out the particles that are exhaled in the collector, thereby exhausting to the exterior nothing but clean filtered air.

In one embodiment, the vapor collector comprises at least one entrance point that is meant to be connected to the second branch of the nozzle and at least one exit point which remains open to the air.

### DETAILED DESCRIPTION

An embodiment of the invention will be described next by way of non-limiting example, with reference to the attached drawings, in which:

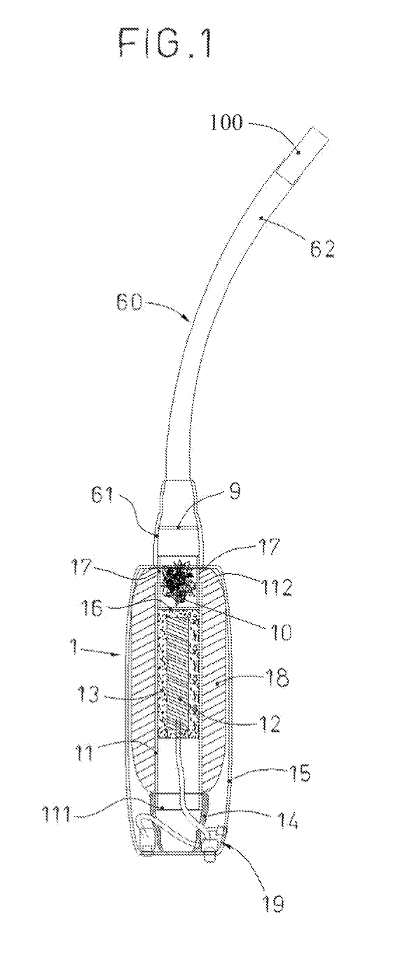

FIG. 1 shows a view of the cross-section of a first embodiment of the essence vaporizer according to the present invention; and

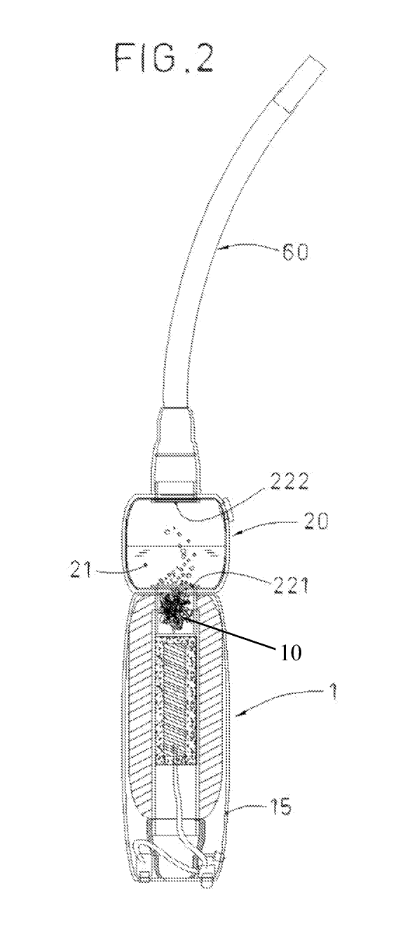

FIGS. 2 to 4 show other alternative embodiments of the essence vaporizer.

The invention provides an essence vaporizer for extracting the essence of a substance using the method of evaporation by displacement of hot air, known as forced thermal convection.

In the embodiment of FIG. 1, the vaporizer comprises a chamber 10 for containing in its interior a substance of which its essence is to be extracted; and a tubular element 11 that, at its center, contains the whole of the heat source 12 and a heat exchanger 13, used to heat up the air in its interior and thus allowing for the essence extraction of the substance contained in the chamber 10 when the user inhales such air.

The tubular element 11 and the chamber 10 are surrounded by an insulation 18. The whole is to be found inside the interior of the casing 15, which is open at its ends and is linked to the inhalation nozzle 60.

In order to extract the essence, the user inhales air through the nozzle 60 optionally with a mask (not shown) generating an air flow that enters the device at the lower end of the casing 15, and goes through the heat exchanger 13, the chamber 10, and the nozzle 60, thus reaching the user.

The casing 15 also works as an adequate handle so that the user can hold the vaporizer in his hands.

In the current embodiment, the thermal insulation 18 is made of a nanoporous material, and more specifically a microporous material, with monolithic structures of metal oxides, amorphous silica and glass filaments, pressed together to form a solid block. This gives the insulation a very low thermal conductivity and a good dimensional stability at high temperatures (with very little contraction in the cooling down process).

The nanoporous material has a pore size inferior to 50 nm., and preferably inferior to 2 nm. (microporous material).

Microporous materials suitable for the insulation 18, as well as their properties and manufacturing processes, are described in the patent applications WO2005/040063 y WO2006/097668, which can be referred to for more information on this matter.

A specific example of a suitable insulation is the so-called MPS®, from Microtherm®.

The thermal insulation 18 is placed surrounding the tubular element 11 and in direct contact with it. Its thickness is so that the external surface area, in contact with the interior of the casing 15, maintains the temperature that allows for the object to be handled without risk of burns or degradation of the casing. The length of the thermal insulation covers at least the contact area between the tubular element 11 and the group formed by the heat source 12 and the heat exchanger 13,

As shown in FIG. 1, the tubular element 11 has a circular or polyhedral cross-section, open at its two ends, which constitute an air entrance point 111 and an air exit point 112. The materials used for the elements found within the tubular element 10 have to withstand the maximum working temperatures of the heat source.

The heat source 12 is a device consisting of electrodes that convert electrical energy into thermal energy. The electric current flow is proportional to the energy transformed into heat. By increasing or decreasing the electrical current with an electric controller 19 we can reach different exit temperatures for the air flow that goes through the exchanger 13. For instance, the heat source can be a FIREROD® cartridge heater manufactured by WATLOW®.

The heat exchanger 13 can be a stainless steel open cell foam heat exchanger, such as the one commercialized by AlCarbon, which has a high specific surface area.

Additionally, its tubular shape is such that the heat source 12 can be inserted in its interior to transmit energy throughout the whole surface area. Both the open cell foam structure and said tubular shape provide a large contact surface area with air that favors heat exchange. In another embodiment, the stainless steel foam could be substituted by a foam of a ceramic material, which has a higher specific heat capacity than steel.

At the air entrance point 111 of the tubular element 11, a tubular profile 14 which is open at its ends, is placed, in a sealing manner joining together the air entrance point 111 of the tubular element 11 and the air entrance of the casing. The power supply cables of the heat source 12 go through the tubular profile without compromising its sealing. The connecting tubular profile 14 could be made of silicone or some other elastomer that guarantees the sealing property and that can withstand the operational temperatures.

Within the tubular element 111, in contact with and facing one of the ends of the heat exchanger 13 neighboring the air exit 112, there is a mesh disc 16 with a mesh opening between 10 and 500 microns and with a cross-section that allows it to be inserted in the tubular element 11 adjusting itself to the internal diameter of the tubular element. In some of the embodiments, the mesh disc is made of stainless steel, preferably with a thread thickness of 200 microns and a mesh opening of 400 microns.

Next, a tubular element with a non-stick wall 17 is placed within the air exit 112 end of the tubular element 11, so that it supports the mesh disc 16 and defining in its interior, the chamber 10 for the substance of which the essence is to be extracted. This element 11 sticks out of the casing 15 in such a way that it makes it possible to mount the next component, in this case the nozzle 60.

The profile of the tubular element with non-stick wall 17 has a shape that allows it to stay firmly fitted pressing against the interior walls of the tubular element 11. In one of the embodiments the element is a circular profile manufactured of a polymer such as ECTFE (HALAR®).

The casing 15 is metallic, of tubular shape, and it contains in its interior the thermal insulation 18, the whole of the tubular element 11, the heat source 12, the heat exchanger 13, and the mesh disc 16. Its air entrance and exit ends are open and remain in touch with the tubular profile 14 and the tubular element with non-stick wall 17 respectively.

The inhalation nozzle 60 is connected to the tubular element of non-stick wall 17. The entrance end 61 of the nozzle 60 can be inserted externally of the tubular element with non-stick wall 17 in such a way that they stay firmly fastened and allows for a good sealing to the extracted vapor. In other embodiments, the entrance end 61 can be inserted internally of the tubular element 17.

In the embodiment shown in FIG. 1, the nozzle 60 consists of a tubular element which changes cross-section from its vapor entrance end 61 to its vapor exit end 62. In its interior, it contains a mesh filter 9 of similar characteristics as the mesh disc 16. The filter 9 serves as a barrier to solid particles so that the substance or substance portions of which the essence is extracted do not enter the user's respiratory ways during inhalation, yet allowing the extracted vapor essence to do so.

The inhalation nozzle 60 can be made of an elastomer material such as for example silicone. The vapor exit end 62 can have a variable length to enhance user convenience and it has an inserted mouthpiece 100 with a pleasant feel to the user's mouth.

FIG. 2 shows an embodiment in which the vaporizer includes furthermore an anti-spillage humidifier 20 that humidifies the vapor. It is placed between the evaporation chamber 10 and the inhalation nozzle 60.

This way, at the vapor exit of the vaporizer as a whole 1, which is the whole group contained within the casing 15 described up to now, an anti-spillage humidifier 20 is placed in series. The humidifier 20 comprises a water container 21 with an air entrance at its lower end, connected to the vapor exit of the vaporizer group 1. It also comprises an air exit in its upper end, connected to the nozzle 60. The entrance and exit of the humidifier 20 are equipped with hydrophobic membranes 221 and 222 that allow the vapor to pass but prevent water spillage from the container 21. The membranes can be substituted by non-return valves that perform the same anti-spillage function.

The air entrance of the humidifier 20 is connected in series to the vapor exit of the vaporizer group 1, as shown in FIG. 2, if one wishes to perform a dry extraction and a subsequent vapor humidification. However, the humidifier 20 can also be placed the other way around, i.e. connecting the air entrance of the vaporizer group 1 to the air exit of the humidifier 20 to establish an extraction with humid air.

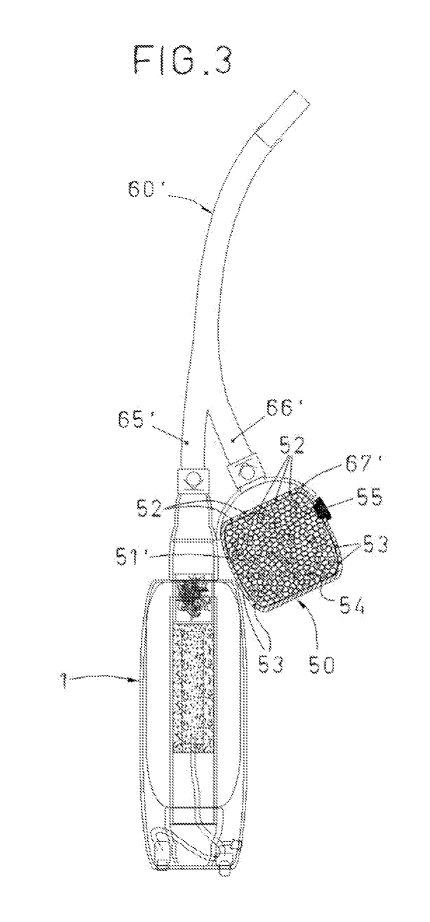

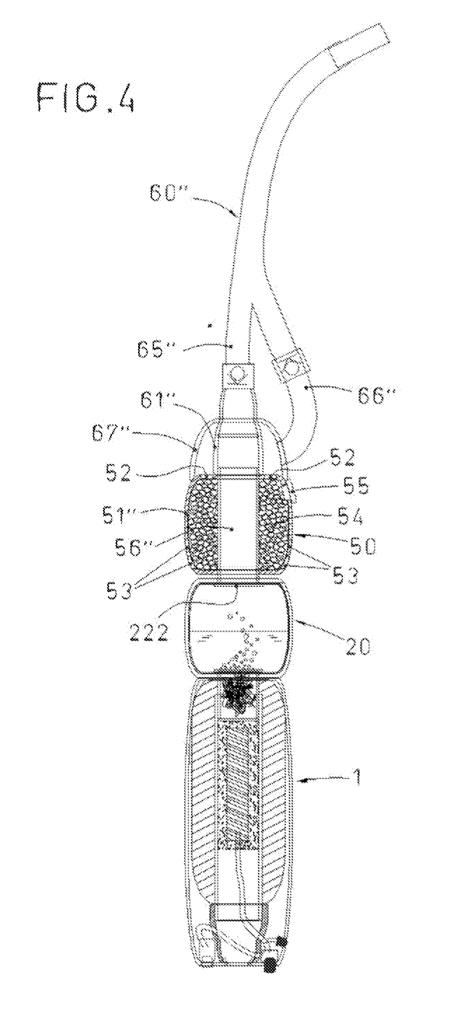

FIGS. 3 and 4 show variations of embodiments which furthermore comprise a vapor collector 50. The purpose of the vapor collector is to filter the air that the user expels into the air after inhaling the essence vapor for example in order to avoid smells.

In order to fit the vapor collector to this type of vaporizer, the inhalation nozzle has to have a different configuration than the nozzle 60 shown in FIG. 1, as will be explained next.

In FIG. 3, a vapor collector 50 comprises a carbon trap 51′ and several air entrance points 52 and air exit points 53. The carbon trap 51′ contains in its interior activated carbon particles 54. It is formed by a container with a mesh opening inferior to the minimum size of the carbon particles.

Also, the nozzle 60′ in FIG. 2 comprises a first branch 65′ coupled to the exit of the vaporizer as a whole 1 and a second branch 66′ connected to a vapor collector 50. The two branches 65′ and 66′ of the nozzle 60′ comprise non-return valves arranged in a suitable way so that the user can inhale the vapor essence through the first branch 65′ and exhale the air through the second branch 66′ all the way to the vapor collector 50. The branch 66′ ends at an adaptor 67′ suitable for being connected to the entrances 52 of the collector 50.

In the vapor collector 50, the vapor expelled by the user and ducted through the branch 65′ flows from the entrances 52 all the way to the exits 53. Along the way it goes through the carbon particles 54, whose purpose is to trap any substance contained in the vapor.

The filling up and emptying of the activated carbon 54 from the interior of the collector 50 can be performed using a plug 55.

In FIG. 4, the vapor collector 50 is similar to that of FIG. 3 but it includes a central duct 56″ so that it can be mounted downstream from the humidifier 20 in the direction of the vapor flow towards the user. As shown in the figure, this central duct 56″ is placed between the membrane 222 of the humidifier 20 and the entrance 61″ of the nozzle 60″, whereas the carbon trap 51″ is configured of annular shape, provided around the duct 56″.

As shown, the nozzle 60″ comprises two branches 65″ and 66″ that include suitable non-return valves. The nozzle is connected to a vapor collector 50 in such a way that the user inhales the vapor with the essence through the first branch 65″, which is communication with the humidifier 20 through the central duct 56″ of the vapor collector 50. The air exhaled by the user goes through the carbon filter of the collector 50.

Both branches 65″ and 66″ of the nozzle 60″ end at an adaptor 67″ that surrounds the lower far end 61″ of the branch 65″ and allows for a suitable fit between the branch 66″ and the entrances 52 of the vapor collector.

The principle of operation of the embodiments in FIGS. 3 and 4 is very similar. The main difference is the mounting system of the vapor collector 50, which in the case of FIG. 4 is better integrated in the device.

As it will be understood from the previous description, in practice the vaporizer comprises a series of modules (vaporizer group, humidifier, vapor collector) that can be connected in different ways, depending on the desired effect for each case. The device can be marketed with different modules and accessories (mouthpieces, adaptors, etc.) so that the user can choose at each moment the configuration that he or she desires.

Notwithstanding the fact that the invention has been described and shown with reference to one or more particular embodiments of the vaporizer, the person skilled in the art will be able to introduce changes and modifications, in accordance with circumstances, and substitute any element for any technical equivalent thereof, without departing from the scope of protection defined by the appended claims.

For instance, the skilled person will understand that although only certain particular embodiments of the vaporizer with humidifier and/or vapor collector have been described, due to the modular build-up of the device, other combinations and configurations are possible.

1. A portable essence vaporizer for extracting an essence and supplying it to a user, which comprises a heat source for heating up an air flow which is passed through a substance from which one can extract an essence, and is supplied to a user, said vaporizer comprising a heat exchanger provided in such a way that the heat generated by the heat source is supplied to the air flow, and a thermal insulation for insulating the heat source and the heat exchanger from the exterior.

2. The vaporizer according to claim 1, wherein the thermal insulation comprises a nanoporous material with pores of a size of less than 50 nm.

3. The vaporizer according to claim 2, wherein the nanoporous material is microporous, with pores of a size of less than 2 nm.

4. The vaporizer according to claim 1, wherein the thermal insulation comprises monolithic structures of metal oxides, amorphous silica and glass filaments.

5. The vaporizer according to claim 1, wherein the heat exchanger comprises an open cell foam structure.

6. The vaporizer according to claim 5, wherein the open cell foam structure has a specific surface area of between 50 and 200 cm²/cm³.

7. The vaporizer according to claim 5, wherein the open cell foam structure is metallic.

8. The vaporizer according to any claim 5, wherein the open cell foam structure is ceramic.

9. The vaporizer according to claim 1, wherein the heat exchanger comprises a tubular piece, the heat source being provided at least partially inside the tubular piece.

10. The vaporizer according to claim 9, wherein the thermal insulation comprises a tubular element which at least partially surrounds the heat exchanger, the thermal insulation, tubular element and heat exchanger being housed within a casing which may be handled by a user.

11. The vaporizer according to claim 1, further comprising a duct for the circulation of the air flow, said duct comprising an extraction chamber for housing the substance being provided downstream of the heat source.

12. The vaporizer according to claim 11, further comprising a humidifier disposed in such a way that the air flow passes through it.

13. The vaporizer according to claim 12, wherein the humidifier is disposed downstream of the extraction chamber so that the air flow passes through it after extracting the essence.

14. The vaporizer according to claim 12, wherein the humidifier is disposed upstream of the extraction chamber, so that the air flow passes through it before extracting the essence.

15. The vaporizer according to claim 1, further comprising an inhalation nozzle through which a user inhales air so as to cause said air flow which passes through the substance from which the essence is to be extracted and which is inhaled by the user.

16. The vaporizer according to claim 15, wherein the inhalation nozzle comprises a first branch through which the air that is inhaled by the user passes and at least a second branch through which the air that is exhaled by the user passes.

17. The vaporizer according to claim 16, further comprising a vapor collector for filtering a vapor exhaled by the user after essence extraction.

18. The vaporizer according to claim 17, wherein the vapor collector comprises activated carbon.

19. Vaporizer according to claim 17, wherein the vapor collector comprises at least an entrance which is to be connected to a second branch of the inhalation nozzle and at least an exit which is to be open to the exterior.
